(19) United States
(12) Patent Application Publication
(10) Pub. No.: US 2025/0304512 A1
(43) Pub. Date: Oct. 2, 2025

(54) SUPPORT ELEMENTS WITH SUPPORT PLATES IN A REACTOR VESSEL

(71) Applicant: L'Air Liquide, Société Anonyme pour I'Etude et I'Exploitation des Procédés Georges Claude, Paris (FR)

(72) Inventors: Tobias Oelmann, Bad Vilbel (DE); Michael Strozyk, Offenbach (DE); Katja Bartels, Frankfurt am Main (DE); Abdullah Enes Tercan, Frankfurt am Main (DE); Veronika Gronemann, Karben (DE)

(21) Appl. No.: 19/090,936

(22) Filed: Mar. 26, 2025

(30) Foreign Application Priority Data
Mar. 28, 2024 (EP) EP24167625

(51) Int. Cl. C07C 1/12; F28D 1/04; F28F 9/013

(52) U.S. Cl. CPC C07C 1/12; F28D 1/0408; F28F 9/0131

(57) ABSTRACT

A device (1.1;1.2) with a reactor vessel (2), a tube bundle (3) of multiple tubes (4), a first support plate (5) and a second support plate (6), wherein the tube bundle (3) is disposed in the reactor vessel (2), wherein the tube bundle (3) comprises a first tube group (7), a second tube group (8) and a third tube group (9), wherein the first support plate (5) and the second support plate (6) are disposed in the reactor vessel (2) transversely to a longitudinal axis (10) of the reactor vessel (2), wherein each of the tubes (4) is made to pass through the first support plate (5) and the second support plate (6).

### CROSS REFERENCE TO RELATED APPLICATIONS

This application claims the benefit of priority under 35 U.S.C. § 119 (a) and (b) to European Patent Application No. 24167625, filed Mar. 28, 2024, the entire contents of which are incorporated herein by reference.

### BACKGROUND

The invention relates to an assembly for methanol synthesis and to a device that can be part of such an assembly. The invention also relates to a use of the device and to a process for methanol synthesis using the device.

Processes for the industrial preparation of methanol by heterogeneously catalysed conversion of synthesis gas in suitable synthesis reactors are known. Synthesis gases may be various gas mixtures which contain hydrogen and carbon oxides, among other things. The reactor is usually in the form of an upright fixed-tube heat exchanger.

Two-stage processes for preparing methanol are also known. In these processes, synthesis gas is fed to a water-cooled reactor and then to a gas-cooled reactor. A copper-based solid-bed catalyst is used to convert the synthesis gas to methanol.

In the case of a water-cooled reactor, the catalyst is inside the tubes, surrounded by water or steam on the shell side. The tubes are mechanically fixed in the reactor via tube plates and supporting metal sheets.

The cooling in the water-cooled reactor is performed via heat being released into the water, whereupon steam can be produced. The steam-water mixture rises on the tubes. The supporting metal sheets must ensure the fixing of the tubes. Supporting metal sheets inserted offset in the reactor vessel, such that the coolant flows in a meandering course in the reactor vessel around the supporting metal sheets, are known.

Depending on the selected geometries of the heat-transferring components and of the mechanical elements of the coolant side, a pressure drop is produced in the steam on the coolant side. It is primarily long reactor tubes which result in a higher pressure drop.

Frequently, the external dimensions of the reactor are restricted for transport, and this leads to long, slender reactors. However, the consequence of this is an increased pressure drop on the coolant side. The long shape of the reactor means further support plates are required to avoid the tubes sagging when they are being transported, and also avoid them bowing, bending and swaying when in operation.

### SUMMARY

An object of the present invention, proceeding from the described prior art, is to reduce the hindering effect of the support plates and at the same time prevent the tubes from bending, bowing or swaying.

This object is achieved by the subject matter of the independent claims. Further advantageous configurations are specified in the dependent claims. The features set out in the claims and in the description may be combined with one another in any technologically appropriate way.

The invention sets out a device which comprises a reactor vessel, a tube bundle of multiple tubes, a first support plate and a second support plate. The tube bundle is disposed in the reactor vessel. The tube bundle comprises a first tube group, a second tube group and a third tube group.

The first support plate and the second support plate are disposed in the reactor vessel transversely to a longitudinal axis of the reactor vessel.

Each of the tubes of the first tube group is routed through a respective tube opening of the first support plate, wherein the first support plate has multiple fluid-exchange apertures. Each of the tubes of the second tube group is routed through a respective one of the apertures in the first support plate.

Each of the tubes of the second tube group is routed through a respective tube opening of the second support plate, wherein the second support plate has multiple fluid-exchange apertures. Each of the tubes of the first tube group is routed through a respective one of the apertures in the second support plate.

Each of the tubes of the third tube group is routed through a respective tube opening of the first support plate and made to pass through the second support plate. The first support plate supports the tubes of the first tube group and the third tube group in the tube openings of the first support plate transversely to the longitudinal direction of the tubes. The second support plate supports the tubes of the second tube group in the tube openings of the second support plate.

The device comprises a reactor vessel and a tube bundle of multiple tubes in the reactor vessel. A first medium can flow through the tubes. Outside the tubes there may be a second medium in the reactor vessel. The second medium may flow through intermediate spaces between the tubes. Between the first medium and the second medium, an exchange of heat can take place. The first medium and the second medium may flow in opposite directions. In this case, the device is operated in a counter-current configuration. The first medium and the second medium may, however, also flow in the same direction. In the case of methanol synthesis, the first medium may for example contain the reaction reactants and the second medium may be a cooling medium, or vice versa.

The reactor vessel is preferably an elongate hollow body. The reactor vessel may be a cylindrical metal vessel suitable for encompassing the tube bundle. The reactor vessel may have ports and connections for fluidically connecting the tube bundle. Furthermore, the reactor vessel may have ports and connections so that a cooling medium or other medium can be introduced into and discharged from the reaction vessel into or from a shell space outside and between the tubes. The shell space may be fluidically connected.

The tube bundle comprises multiple tubes. The tube bundle preferably comprises at least 6 tubes. The tube bundle more preferably comprises at least 16 tubes. The tube bundle particularly preferably comprises even at least 1000 tubes.

The tube bundle comprises a first tube group, a second tube group and a third tube group. Each of the tube groups comprises in each case several of the tubes. It is possible, but not necessary, that the tube bundle also includes one or more tubes that do not belong to any of the tube groups. Each of the tubes belongs either to the first tube group, to the second tube group, to the third tube group or to none of the tube groups. Even if there are tubes that belong to none of the tube groups, there is in any case no tube which belongs to more than one of the tube groups at the same time. The tube groups are defined only by the properties described herein. It is not necessary for the assignment of the tubes to the tube groups also to be evident from structural features of the device.

Preferably, the first tube group comprises at least six tubes and/or the second tube group comprises at least six tubes and/or the third tube group comprises at least two tubes. Particularly preferably, the number of tubes of the first tube group is in the ratio 3:1 to the number of tubes of the third tube group and/or the number of tubes of the second tube group is in the ratio 3:1 to the number of tubes of the third tube group. The “and” versions are preferred.

The tubes may be elongate cylindrical hollow bodies with a uniform wall thickness. The wall thickness of the tubes is preferably at most 5 mm, more preferably at most 2.5 mm and particularly preferably at most 0.1 mm. In particular, the wall thickness of the tubes may correspond to a wall thickness which is standard for the application. The tubes may be made in particular of a metallic material. The material preferably conducts heat.

Some of the tubes or all of the tubes preferably contain a catalyst. In the case of methanol synthesis, it is thus possible for example to conduct the reaction reactants through the tubes and react them with the catalyst in the tubes so as to form methanol.

The reaction reactants may be provided in particular in the form of a synthesis gas. Preferred reaction reactants are hydrogen, carbon monoxide and carbon dioxide. A mixture of hydrogen and carbon monoxide is preferred. A mixture of hydrogen and carbon dioxide is particularly preferred. Furthermore, the synthesis gas may contain inert gases. In particular, the synthesis gas may contain methane as an inert gas. The synthesis gas preferably contains nitrogen as an inert gas. The synthesis gas can be conducted into the tubes on an inlet side and react with the catalyst in the tube. In the case of an exothermic reaction, heat generated during the reaction can be released via the tube shell. A coolant can discharge the heat, preferably by convection, from the shell side. The reaction product formed in the tube can be discharged, together with the remaining synthesis gas, on the outlet side.

As an alternative, a synthesis gas with the reaction reactants can also be conducted through the reaction vessel outside the tubes. In particular in this case, a catalyst bed may be provided on the shell side in the shell space. The synthesis gas can then be passed through the catalyst bed. A coolant can be conducted into the tubes on an inlet side. The heat generated by the reaction can be transferred to the coolant in the tubes via the shell side of the tubes. The heated coolant can be discharged on an outlet side of the tubes.

Furthermore, the device comprises a first support plate and a second support plate. The first support plate and/or the second support plate may be in the form of a metal sheet. The first support plate and/or the second support plate preferably have a thickness of at least 7 mm. The thickness of the first support plate and/or of the second support plate is preferably at most half of a diameter of the tubes. The optimum thickness of the support plate can also be ascertained via calculations. If not all the tubes have the same diameter, the thickness of the first support plate and/or of the second support plate is preferably at most half of the largest diameter of the tubes of the tube bundle.

The first support plate and the second support plate are disposed in the reactor vessel transversely to a longitudinal axis of the reactor vessel. In particular, the first support plate and the second support plate may be fastened in place in the reactor vessel.

The longitudinal axis of the reactor vessel is preferably aligned along the elongate direction of the hollow body. In particular, the first support plate and/or the second support plate is disposed in the reactor vessel perpendicularly to the longitudinal axis of the reactor vessel. The reactor vessel and/or the tube bundle is preferably vertically aligned. The “and” versions are preferred.

The first support plate is preferably at a spacing of at most 500 mm from the second support plate along the longitudinal axis of the reactor vessel.

Each of the tubes of the first tube group is routed through a respective tube opening of the first support plate, wherein the first support plate has multiple fluid-exchange apertures. Each of the tubes of the second tube group is routed through a respective one of the apertures in the first support plate.

Each of the tubes of the second tube group is routed through a respective tube opening of the second support plate, wherein the second support plate has multiple fluid-exchange apertures. Each of the tubes of the first tube group is routed through a respective one of the apertures in the second support plate.

Each of the tubes of the third tube group is routed through a respective tube opening of the first support plate and made to pass through the second support plate.

The tube openings of the first support plate are assigned to the tubes of the first and third tube groups. The tube openings of the second support plate are assigned to the tubes of the second tube group.

The first support plate and the second support plate may adversely affect the fluid exchange in the shell space of the reactor vessel. In order to keep this effect as small as possible, the first support plate and the second support plate each have multiple apertures. The fluid exchange takes place in particular between reactor-vessel regions between which the first support plate and the second support plate are disposed.

A free flow cross section in the reactor vessel can be determined by the apertures in the first support plate and in the second support plate. The free flow cross section is that cross section of a tube or channel through which a medium flows. The free flow cross section thus denotes an area through which a medium can flow. The greater the smallest overall area of all the apertures of the first support plate or of the second support plate, the greater the free flow cross section. On the other hand, the free flow cross section may also be determined via the pitch. The pitch describes the distance from tube to tube. The greater the distance between tubes, the greater the free flow cross section without a support plate. The pitch can thus define a maximum area of the apertures.

The first support plate supports the tubes of the first tube group and the third tube group in the tube openings of the first support plate transversely to the longitudinal direction of the tubes. The second support plate supports the tubes of the second tube group in the tube openings of the second support plate.

The tubes of the first tube group are routed through a respective tube opening of the first support plate. Each tube has a dedicated tube opening. As a result, the tubes of the first tube group are supported by the first support plate. The tubes of the first tube group are moreover routed through a respective aperture of the second support plate. The first tube group comprises all the tubes that are routed through one of the apertures of the second support plate. The tubes of the first tube group are in general not supported by the second support plate. Instead, these tubes pass through the second support plate merely by being routed through the corresponding apertures. The apertures in the second support plate are used for fluid exchange.

The tubes of the second tube group are routed through a respective tube opening of the second support plate. Each tube has a dedicated tube opening. As a result, the tubes of the second tube group are supported by the second support plate. The tubes of the second tube group are moreover routed through a respective aperture of the first support plate. The second tube group thus comprises in each case all the tubes that are together routed through one of the apertures of the first support plate. The tubes of the second tube group are in general not supported by the first support plate. Instead, these tubes pass through the first support plate merely by being routed through the corresponding apertures. The apertures in the first support plate are used for fluid exchange.

The tubes of the third tube group are routed through a respective tube opening of the first support plate and made to pass through the second support plate. Each tube of the third tube group has a dedicated tube opening in the first support plate. As a result, the tubes of the third tube group are supported by the first support plate.

This has the advantage that the tubes of the tube bundle can individually thermally expand, but vibrations caused by certain flow states and bending of the tubes can be avoided. A further advantage is that all the tubes of the tube bundle are supported by a first support plate together with a second support plate.

The fact that the tubes of the third tube group are made to pass through the second support plate can be realized in various ways. For example, the tubes of the third tube group may be routed through a respective tube opening in the second support plate, wherein the second support plate supports the tubes of the third tube group in the tube openings of the second support plate. Alternatively, the tubes of the third tube group may be routed through a respective fluid-exchange aperture in the second support plate. Also, several or all of the tubes of the third tube group may together be routed through a fluid-exchange aperture in the second support plate.

The tubes of the tube bundle are preferably not axially fixed in place in the respective tube opening of the first support plate and of the second support plate. The tubes of the first tube group and the third tube group are in this case axially movable in the axial direction through a respective tube opening of the first support plate. The tubes of the second tube group are in this same case axially movable in the axial direction through a respective tube opening of the second support plate. Locally different temperatures can result in locally different expansions of the tubes. Since the tubes are axially movable, individual tubes are not disrupted in their individual expansion by one of the support plates. Movements transverse to the longitudinal axis are, however, minimized.

The shape of the first support plate and of the second support plate can be adapted to that of the reactor vessel. The first support plate and/or the second support plate preferably has a rectangular shape with rounded corners. The first support plate and/or the second support plate particularly preferably has an elliptical shape, in particular a circular shape. The “and” versions are preferred.

The device has the advantage that the hindering effect of the first support plate and of the second support plate is minimized and at the same time the tubes are prevented from bending, bowing or swaying during transport or during operation.

In a preferred embodiment of the device, the tube openings of the first support plate are each surrounded by an annular divider, wherein the dividers each have a minimum width which is the same for at least some of the dividers. In addition or alternatively, the tube openings of the second support plate are each surrounded by an annular divider, wherein the dividers each have a minimum width which is the same for at least some of the dividers.

The first support plate and the second support plate should on the one hand be stable enough to sufficiently support the tubes. This can be achieved by the dividers having a sufficient width. On the other hand, the support plate should adversely affect the fluid exchange as little as possible. This can be achieved by the dividers being kept as small as possible. Assessing the two aforementioned conditions will make it possible to determine how wide a divider should optimally be at its narrowest point, and thus how large the minimum width of a divider should optimally be. In the present embodiment, at least some of the dividers, preferably even all the dividers, have the same minimum width. In this way, the best possible compromise between stability and fluid exchange can be achieved for all these dividers.

Preferably, the annular dividers of the first support plate are connected via a respective first transition and/or the annular dividers of the second support plate are connected via a respective second transition. The “and” version is preferred.

In a further preferred embodiment of the device, each of the tubes of the third tube group is routed through a respective tube opening of the second support plate which is congruent to the tube opening of the first support plate through which the respective tube of the third tube group is routed.

The second support plate supports in particular the tubes of the second tube group and the third tube group in the tube openings of the second support plate transversely to the longitudinal direction of the tubes. As a result, the tube openings of the second support plate are assigned to the tubes of the second and third tube groups.

The tubes of the third tube group are routed through a respective tube opening of the first support plate. Each tube has a dedicated tube opening. As a result, the tubes of the third tube group are supported by the first support plate. Furthermore, the tubes of the third tube group are routed through a respective tube opening of the second support plate. Each tube has a dedicated tube opening. As a result, the tubes of the third tube group are also supported by the second support plate. The third tube group thus comprises in each case all the tubes which are routed both through a tube opening in the first support plate and through a tube opening in the second support plate.

This results in a force coupling from the first tube group to the second tube group via the third tube group with the aid of the first support plate and the second support plate.

The advantage of this embodiment is that the tubes of the first tube group and the tubes of the second tube group are mechanically coupled via the tubes of the third tube group. This results in increased stability of the tube bundle in the reactor vessel and better damping properties with respect to vibrations of the tubes during operation. Furthermore, bending of the tubes during transport of the reactor vessel can be more advantageously prevented.

In a further preferred embodiment of the device, the apertures of the first support plate each have a star shape with six prongs. In addition or alternatively, the apertures of the second support plate each have a star shape with six prongs.

This embodiment has the advantage that the free flow cross section is thus as large as possible. This has a positive influence on the hindering effect of the first support plate and the second support plate. As a result, the pressure drop can be reduced.

In a further preferred embodiment of the device, each tube of the third tube group is surrounded in each case by three tubes of the first tube group. In addition or alternatively, each tube of the third tube group is surrounded in each case by three tubes of the second tube group.

This refers in particular to the nearest tubes of the respective tube groups in each case. This described grouping of tubes of the first tube group, the second tube group and the third tube group can also be referred to as a pattern. This pattern may be repeated in the tube bundle.

With the aid of this pattern, forces transverse to the longitudinal axis which act on one of the tube groups can be uniformly dissipated and distributed according to the force coupling described from the first tube group via the third tube group to the second tube group and vice versa.

This allows a more uniform distribution of forces transverse to the longitudinal axis due to, for example, flows or gravitational force during the transport of the reactor vessel.

In a further preferred embodiment of the device, the first support plate and the second support plate together form a support element.

Each of the tubes is thus supported by the first support plate and by the second support plate. The first support plate and the second support plate are to this extent understood as a support element. The support element thus brings about the desired support of all the tubes. Preferably, the first support plate is disposed directly on the second support plate. The described advantages provided by the first support plate together with the second support plate are already achieved when the device has a single support element.

In a preferred development of the preceding embodiment, several of the support elements are disposed in the reactor vessel, offset in relation to one another along the longitudinal axis of the reactor vessel.

However, it is also possible and even preferable for the device to have multiple support elements, which each have a first support plate and a second support plate. The support elements are preferably each designed as described herein.

The advantage of multiple support elements is that the tubes of the tube bundle are held particularly securely. Bending, bowing or swaying of the tubes is advantageously reduced even further.

A further aspect of the invention sets out an assembly for methanol synthesis. The assembly comprises the described device. The described advantages and features of the device can be applied and transferred to the assembly.

To produce methanol, a synthesis gas can be converted by reaction over catalysts to yield methanol. The synthesis gas can contain reaction reactants for the methanol synthesis.

Besides the described device, the assembly preferably comprises a source for reaction reactants for the methanol synthesis. The assembly may also comprise multiple devices designed as described and for example connected in series.

A further aspect of the invention sets out a process. In the process, reaction reactants for the methanol synthesis are conducted through the tubes of the tube bundle. A cooling medium is conducted through the reactor vessel outside the tubes of the tube bundle. The cooling medium is preferably liquid water and/or steam.

In an alternative embodiment of the process, the reaction reactants for the methanol synthesis are conducted through the reactor vessel around the tubes of the tube bundle. A cooling medium is then conducted through the tubes of the tube bundle. The cooling medium is preferably gaseous.

The described advantages and features of the device can be applied and transferred to the process, and vice versa. The device is preferably designed for operation according to the described process. The process is preferably performed with the device.

A further aspect of the invention sets out a use. The described device is used for methanol synthesis. Reaction reactants are converted to methanol in the device.

The described advantages and features of the device, the assembly and the process can be applied and transferred to the use, and vice versa.

The device is preferably used for water-cooled methanol synthesis. As an alternative, it is preferred to use the device for gas-cooled methanol synthesis.

In water-cooled methanol synthesis, the tubes are cooled with water, while a synthesis gas is converted to methanol in the tubes. The water discharges the heat from the tubes and partially evaporates. The rising bubbles of steam can flow through the apertures of the first support plate and through the apertures of the second support plate, or vice versa.

In gas-cooled methanol synthesis, the tubes are cooled by a cooler gas in the tubes. The synthesis gas is converted to methanol outside the tubes. The heat is transferred to the tubes and discharged by the gas in the tubes. The synthesis gas can flow through the apertures of the first support plate and through the apertures of the second support plate, or vice versa.

### BRIEF DESCRIPTION OF THE DRAWINGS

The invention is elucidated in detail hereinafter with reference to the figures. The figures show a particularly preferred exemplary embodiment, but the invention is not limited thereto. The figures and the size ratios represented therein are merely schematic. In the figures:

FIG. 1: shows a schematic view of an assembly according to the invention for methanol synthesis,

FIG. 2: shows a perspective view of a detail of a first configuration of a first support plate and a second support plate, as can be used in the assembly from FIG. 1.

### DETAILED DESCRIPTION OF THE PREFERRED EMBODIMENTS

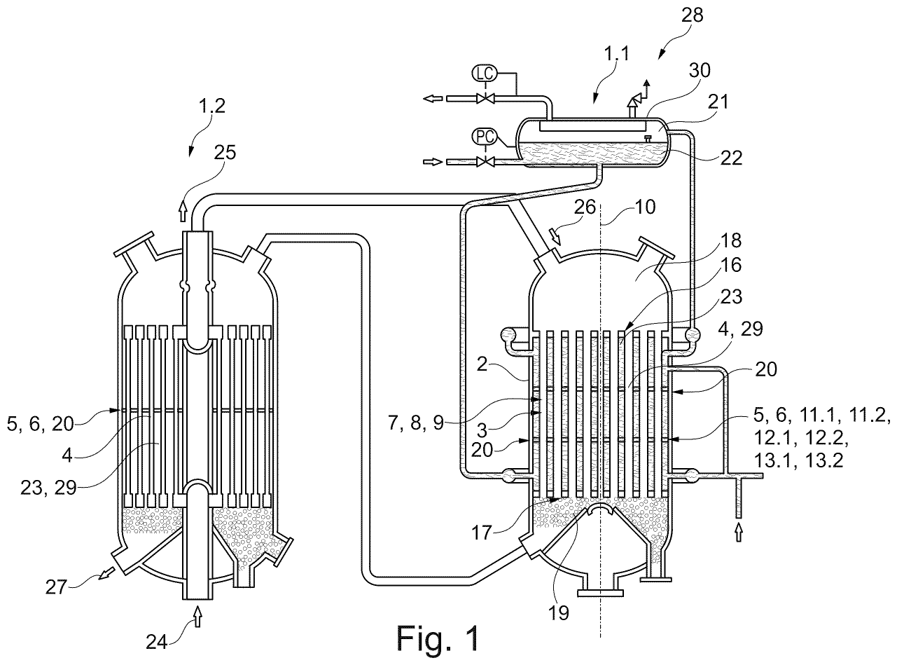

FIG. 1 shows a schematic view of an assembly 28 according to the invention for methanol synthesis. Synthesis gas is conducted through two devices 1.1;1.2 connected in series. The devices 1.1;1.2 are used for methanol synthesis. Pre-heated synthesis gas is fed to the first device 1.1 through a gas inlet 26.

The first device 1.1 comprises a reactor vessel 2, a tube bundle 3 of multiple tubes 4, a first support plate 5 and a second support plate 6. The tube bundle 3 is disposed in the reactor vessel 2, wherein the tube bundle 3 comprises a first tube group 7, a second tube group 8 and a third tube group 9. The first support plate 5 and the second support plate 6 are disposed in the reactor vessel 2 transversely to a longitudinal axis 10 of the reactor vessel 2. Each of the tubes 4 of the first tube group 7 is routed through a respective tube opening 11.1 of the first support plate 5, and the first support plate 5 has multiple fluid-exchange apertures 13.1. Each of the tubes of the second tube group 8 is routed through a respective one of the apertures 13.1 in the first support plate 5, wherein each of the tubes 4 of the second tube group 8 is routed through a respective tube opening 12.1 of the second support plate 6, and wherein the second support plate 6 has multiple fluid-exchange apertures 13.2, wherein each of the tubes of the first tube group 7 is routed through a respective one of the apertures 13.2 in the second support plate 6, wherein each of the tubes 4 of the third tube group 9 is routed through a respective tube opening 11.2 of the first support plate 5, wherein the first support plate 5 supports the tubes 4 of the first tube group 7 and the third tube group 9 in the tube openings 11.1, 11.2 of the first support plate 5 transversely to the longitudinal direction of the tubes 4 and the second support plate 6 supports the tubes 4 of the second tube group 8 in the tube openings 12.1 of the second support plate 6.

Each of the tubes 4 of the third tube group 9 is routed through a respective tube opening 12.2 of the second support plate 6 which is congruent to the tube opening 11.2 of the first support plate 5 through which the respective tube 4 of the third tube group 9 is routed.

The first support plate 5 and the second support plate 6 together form a support element 20. Several of the support elements 20 are disposed in the reactor vessel, offset in relation to one another along the longitudinal axis 10 of the reactor vessel 2.

The tubes 4 are connected at a first end 16 and at a second end 17 to a respective tube end plate. The reactor vessel 2 and the tubes 4 of the tube bundle 3 are upright. The first end 16 is arranged at the top and the second end 17 at the bottom of the reactor vessel 2. The synthesis gas is distributed in a distributor 18 among the tubes 4 of the tube bundle 3.

The tubes 4 contain a catalyst 29. The synthesis gas contains reaction reactants. The reaction reactants for the methanol synthesis are conducted through the tubes 4 of the tube bundle 3. The synthesis gas in the tubes 4 is partially converted to methanol. A cooling medium is conducted through the reactor vessel 2 outside the tubes 4 of the tube bundle 3. The heat released from the exothermic reaction in the tube 4 is transferred via the tube wall to the cooling medium located in a shell space 23. While the catalyst 29 in the tube 4 is cooled by this process, energy is fed to the cooling medium. The cooling medium used is water, which is fed to the shell space 23 at the second end 17. Resulting bubbles of steam and the boiling two-phase water mixture flow vertically upwards owing to differences in density. The steam flows through the apertures 13.1 of the first support plate 5 and through the apertures 13.2 of the second support plate 6 with small pressure drops. The steam is collected at the top and fed to a water condenser 30. The reservoir of the condenser 30 contains saturated steam 21 and water 22 at or slightly below boiling point. The water 22 is fed to the first device 1.1 at the second end 17 and distributed throughout the shell space 23 of the reactor vessel 2. The cooling thus works according to the thermo-siphon effect.

Product gas and the partially unreacted synthesis gas are collected from the tube bundle 3 in a collector 19. The gas mixture then flows to the second device 1.2.

In the second device 1.2, which is downstream of the first device 1.1, synthesis gas from a synthesis gas inlet 24 is pre-heated.

The second device 1.2 has a partially similar structure to the first device 1.1.

By contrast to the first device 1.1, the second device 1.2 contains, between the tubes 4 of the second device 1.2 in the shell space 23, a catalyst 29 in the form of a catalyst bed.

The synthesis gas, which comprises the reaction reactants, is conducted through the reactor vessel 2 through the shell space 23 for the methanol synthesis outside the tubes 4 of the tube bundle 3 and a cooling medium is conducted through the tubes 4 of the tube bundle 3. The synthesis gas in the shell space 23 flows downwards to a product gas outlet 27 with small pressure drops through the apertures 13.1 of the first support plate 5 and through the apertures 13.2 of the second support plate 6.

The heat released from the exothermic reaction in the shell space 23 is transferred via the tube wall to the cooling medium located in the tube 4. While the catalyst 29 in the shell space 23 is cooled by this process, energy is fed to the cooling medium. The cooling medium used is synthesis gas, which is fed to the tubes 4 via the synthesis gas inlet 24. The energy fed in causes the synthesis gas from the synthesis gas inlet 24 to be heated and it is passed in the form of pre-heated synthesis gas 25 to the gas inlet 26 of the first device 1.1.

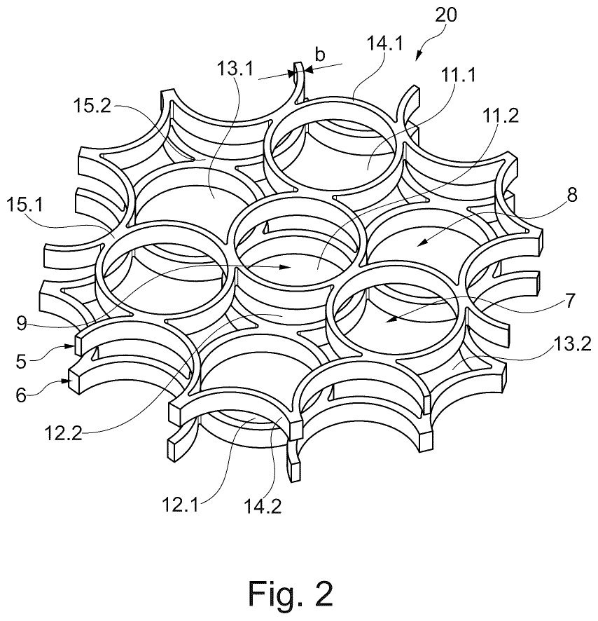

FIG. 2 shows a perspective view of a detail of a first configuration of a first support plate 5 and a second support plate 6, as can be used in the assembly 28 from FIG. 1.

The tube openings 11.1,11.2 of the first support plate 5 are each surrounded by an annular divider 14.1, wherein the dividers 14.1 each have a minimum width b which is the same for the dividers 14.1. The tube openings 12.1, 12.2 of the second support plate 6 are each surrounded by an annular divider 14.2, wherein the dividers 14.2 each have a minimum width b which is the same for the dividers 14.2.

Each of the tubes 4 of the third tube group 9 is routed through a respective tube opening 12.2 of the second support plate 6 which is congruent to the tube opening 11.2 of the first support plate 5 through which the respective tube 4 of the third tube group 9 is routed. The tubes 4 of the first tube group 7, the second tube group 8 and the third tube group 9 are not shown.

The apertures 13.1 of the first support plate 5 each have a star shape with six prongs and the apertures 13.2 of the second support plate 6 each have a star shape with six prongs.

Each tube 4 of the third tube group 9 is surrounded in each case by three tubes 4 of the first tube group 7 and each tube 4 of the third tube group 9 is surrounded in each case by three tubes 4 of the second tube group 8.

Accordingly, each of the tube openings 11.2 of the first support plate 5 is surrounded in each case by three tube openings 11.1 of the first support plate 5 and each of the tube openings 12.2 of the second support plate 6 is surrounded in each case by three tube openings 12.1 of the second support plate 6.

The first support plate 5 and the second support plate 6 together form the support element 20.

The annular dividers 14.1 of the first support plate 5 are connected via a respective first transition 15.1 and the annular dividers 14.2 of the second support plate 6 are connected via a respective second transition 15.2.

The advantage is that the tubes 4 of the first tube group 7 and the tubes 4 of the second tube group 8 are mechanically coupled via the tubes 4 of the third tube group 9. This results in increased stability of the tube bundle 3 in the reactor vessel 2 and better damping properties with respect to vibrations of the tubes 4 during operation. Furthermore, bending of the tubes during transport of the reactor vessel 2 can be more advantageously prevented. Furthermore, the hindering effect is minimized by this configuration of the first support plate 5 and the second support plate 6.

### LIST OF REFERENCE NUMERALS

1.1, 1.2 Device
2 Reactor vessel
3 Tube bundle
4 Tubes
5 First support plate
6 Second support plate
7 First tube group
8 Second tube group
9 Third tube group
10 Longitudinal axis
11.1, 11.2 Tube opening of the first support plate
12.1, 12.2 Tube opening of the second support plate
13.1, 13.2 Aperture
14.1, 14.2 Divider
15.1, 15.2 Transition
16 First end
17 Second end
18 Distributor
19 Collector
20 Support element
21 Saturated steam
22 Water slightly below or at boiling point
23 Shell space
24 Synthesis gas inlet
25 Pre-heated synthesis gas
26 Gas inlet
27 Product gas outlet
28 Assembly
29 Catalyst
Condenser
b Minimum width

1. A device (1.1;1.2) comprising a reactor vessel (2), a tube bundle (3) of multiple tubes (4), a first support plate (5) and a second support plate (6),
wherein the tube bundle (3) is disposed in the reactor vessel (2),
wherein the tube bundle (3) comprises a first tube group (7), a second tube group (8) and a third tube group (9),
wherein the first support plate (5) and the second support plate (6) are disposed in the reactor vessel (2) transversely to a longitudinal axis (10) of the reactor vessel (2),
wherein each of the tubes (4) of the first tube group (7) is routed through a respective tube opening (11.1) of the first support plate (5), and wherein the first support plate (5) has multiple fluid-exchange apertures (13.1), wherein each of the tubes (4) of the second tube group (8) is routed through a respective one of the apertures (13.1) in the first support plate (5),
wherein each of the tubes (4) of the second tube group (8) is routed through a respective tube opening (12.1) of the second support plate (6), and wherein the second support plate (6) has multiple fluid-exchange apertures (13.2), wherein each of the tubes of the first tube group (7) is routed through a respective one of the apertures (13.2) in the second support plate (6),
wherein each of the tubes (4) of the third tube group (9) is routed through a respective tube opening (11.2) of the first support plate (5) and is made to pass through the second support plate (6),
wherein the first support plate (5) supports the tubes (4) of the first tube group (7) and the third tube group (9) in the tube openings (11.1, 11.2) of the first support plate (5) transversely to the longitudinal direction of the tubes (4) and the second support plate (6) supports the tubes (4) of the second tube group (8) in the tube openings (12.1) of the second support plate (6).

2. The device (1.1;1.2) according to claim 1, wherein the tube openings (11.1,11.2) of the first support plate (5) are each surrounded by an annular divider (14.1), wherein the dividers (14.1) each have a minimum width (b) which is the same for at least some of the dividers (14.1), and/or
the tube openings (12.1) of the second support plate (6) are each surrounded by an annular divider (14.2), wherein the dividers (14.2) each have a minimum width (b) which is the same for at least some of the dividers (14.2).

3. The device (1.1;1.2) according to claim 1, wherein each of the tubes (4) of the third tube group (9) is routed through a respective tube opening (12.2) of the second support plate (6) which is congruent to the tube opening (11.2) of the first support plate (5) through which the respective tube (4) of the third tube group (9) is routed.

4. The device (1.1;1.2) according to claim 1, wherein the apertures (13.1) of the first support plate (5) each have a star shape with six prongs and/or
the apertures (13.2) of the second support plate (6) each have a star shape with six prongs.

5. The device (1.1;1.2) according to claim 1, wherein each tube (4) of the third tube group (9) is surrounded in each case by three tubes (4) of the first tube group (7) and/or each tube (4) of the third tube group (9) is surrounded in each case by three tubes (4) of the second tube group (8).

6. The device (1.1;1.2) according to claim 1, wherein the first support plate (5) and the second support plate (6) together form a support element (20).

7. The device (1.1;1.2) according to claim 6, wherein several of the support elements (20) are disposed in the reactor vessel, offset in relation to one another along the longitudinal axis (10) of the reactor vessel (2).

8. A process for methanol synthesis using the device (1.1; 1.2) according to claim 1, wherein reaction reactants for the methanol synthesis are conducted through the tubes (4) of the tube bundle (3) and a cooling medium is conducted through the reactor vessel (2) outside the tubes (4) of the tube bundle (3), or
wherein reaction reactants for the methanol synthesis are conducted through the reactor vessel (2) outside the tubes (4) of the tube bundle (3) and a cooling medium is conducted through the tubes (4) of the tube bundle (3).
